(19) United States
(12) Patent Application Publication
(10) Pub. No.: US 2003/0082215 A1
(43) Pub. Date: May 1, 2003

(54) Fenofibrate galenic formulations and method for obtaining same

(76) Inventors: Josiane Lemut, Dijon (FR); Pascale Blouquin, Talant (FR); Philippe Reginault, Fontaine-Les-Dijon (FR)

Correspondence Address: MERCHANT & GOULD PC, P.O. BOX 2903, MINNEAPOLIS, MN, 55402-0903, US

(21) Appl. No.: 10/168,552

(22) PCT Filed: Dec. 29, 2000
(86) PCT No.: PCT/FR00/03738

(30) Foreign Application Priority Data
Dec. 31, 1999 (FR) 99/16807

(51) Int. Cl. A61K 9/48; A61K 31/355; A61K 31/235

(52) U.S. Cl. 424/400; 514/458; 514/545

(57) ABSTRACT

The present invention relates to a pharmaceutical composition for the oral administration of fenofibrate in the form of a preconcentrate capable of forming an oil-in-water microemulsion spontaneously on contact with an aqueous medium, of the type comprising:

a lipophilic phase preferably comprising an oil based on glycerol or propylene glycol esters; and

an emulsifying system comprising:

a lipophilic surfactant; and

a hydrophilic co-surfactant.

According to the invention, this composition is characterized in that it also comprises vitamin E acetate in a sufficient amount to stabilize said preconcentrate without the incorporation of an additional hydrophilic component, and makes it possible to prepare novel drugs in the form of sealed gelatin capsules or soft capsules.

The present invention relates to novel galenical fenofibrate formulations for oral administration, to the process for their preparation and to the drugs manufactured from these formulations.

Fenofibrate (INN) is a medicinal active principle which has been known for many years for its efficacy in lowering blood triglyceride and cholesterol levels. Thus fenofibrate is widely prescribed in numerous countries when it is necessary to reduce the risk of atherogenesis.

It is also known that, to obtain a satisfactory hypocholesterolemic effect, it is desirable to maintain a blood level of fenofibric acid (which is the active metabolite of fenofibrate) in the order of 6 to 10 mg/l. Such a level is obtained particularly with a unit dose of 300 mg of fenofibrate in gelatin capsule form (cf. Drugs 40 (2) pp. 260-290 (1990)).

It is also known that substantial variations in blood levels exist according to the pathological conditions observed in the patients. In general terms, for all drugs, it is preferable to maintain the blood level of active metabolite which is necessary to achieve the desired therapeutic effect while minimizing the amount of active principle absorbed by the patient. It is for this reason that formulations with the highest possible bioavailability are sought in order to optimize the dosage and limit any side effects of the active principle.

Finally, it is known that the bioavailability of active principles (administered orally) can vary according to whether the drug is absorbed on an empty stomach, during meals or after meals (cf., for example, Int. Journal of Clinical Pharmacology and Biopharmacy 16 pp. 570-574 (1978), Europ. Journal of Clinical Pharmacology 17 pp. 459-463 (1980), Drug Metabolism and Disposition 6 pp. 302-309 (1988)).

In view of these factors, it can be seen that the galenical formulation of an active principle for oral administration is of great importance for obtaining the therapeutic effect under optimal conditions.

Fenofibrate was originally marketed in the form of gelatin capsules containing a 100 mg dose of active principle, with a dosage of 3 capsules per day, and then in the form of gelatin capsules containing a 300 mg dose of active principle, prescribed at a rate of one capsule per day. The studies cited above state that, after the administration of a gelatin capsule containing a 300 mg dose to healthy volunteers, the bioavailability is in the order of 30% with a maximum blood fenofibric acid level of about 6 to 9 mg/l and an area under the curve of 145 to 170 mg/1l.h.

Another formulation disclosed in document EP 330 532 and marketed in France under the mark LIPANTHYL 200M results from a process in which fenofibrate is co-micronized with a solid surface-active compound to give an intimate and finely divided mixture of the two products. This type of formulation makes it possible to reduce the dosage to 200 mg/day in a single dose to give plasma fenofibric acid concentrations very similar to those obtained with a 300 mg dose of non-co-micronized fenofibrate (Journal International de Médecine (1991) no. 206 pp. 48-50). This formulation thus corresponds to an improvement in bioavailability in the order of 30%, based on the original formulation.

Another type of formulation was proposed in document FR 2 494 112. This formulation consists of microgranules in which a neutral core is coated with inicronized fenofibrate and then covered with a protective layer. The recommended dosage in this case is 250 mg/day, which corresponds to an intermediate bioavailability compared with the previous formulations.

More recently, document FR 2 758 459 proposed a composition in the form of granules or tablets in which fenofibrate, in micronized form, is associated with a hydrophilic polymer (especially polyvinylpyrrolidone) and optionally with a surfactant. The results obtained with granules corresponding to this formulation containing a surfactant show that the fenofibrate dissolves more rapidly in laboratory tests. A comparative pharmacokinetic study also shows an improved bioavailability, especially when considering the parameters of plasma fenofibric acid concentration and area under the curve.

Also, document EP 757 911 discloses a process for the preparation of a fenofibrate formulation which consists in preparing a solution of the active principle in diethylene glycol monoethyl ether and filling this solution into soft capsules. According to the results mentioned in said document, the administration of 100 mg/day of fenofibrate affords the plasma fenofibric acid concentrations which are required to assure the efficacy of the drug. However, there is the problem which results from the oral administration of a relatively large amount of solvent. In fact, according to the formulations described, a 100 mg dose of fenofibrate corresponds to a simultaneous dose of 1500 mg of a diethylene glycol ether. Now, as fenofibrate is a hypolipidemic, the drug is intended to be prescribed over prolonged periods, for example at a rate of 100 mg/day if the bioavailability of this formulation has been doubled relative to that of the composition described in document EP 330 532. Thus the use of this formulation would amount to a regular daily absorption of 1.5 g of a diethylene glycol ether, the biological effects of which are not totally neutral (cf. Food Cosmet. Toxicol. (1968) 6 (6) pp. 689-705. Arzneim. Forsch. (1978) 28 (9) pp. 1571-1579; Occup. Hyg. (1996), 2 (1-6, Proceedings of the Int. Symposium on Health Hazards of Glycol Ethers, 1994), 131-151).

Also, 5^{ème} Congrès Intern. Technol. Pharm. vol. 3 (1989) pp. 190-199, discloses a formulation presented in the form of a solution of fenofibrate in dimethyl isosorbide mixed with a dispersant. According to the assays mentioned, the bioavailability obtained is essentially much less dependent on the presence of food in the gastric medium, which already constitutes an advance compared with the conventional gelatin capsule form, whose bioavailability varies from 26% on an empty stomach to 89% after meals.

A similar formulation to the previous one is proposed in document EP 904 781, which recommends the preparation of a co-fused mixture of fenofibrate and a solid dispersant such as croscarmellose or polyvinylpyrrolidone.

It is further known, in the art of galenics, that it is possible to obtain intimate mixtures of a normally water-insoluble, lipophilic oily phase and an aqueous or hydrophilic phase; these mixtures can take the form of a dispersion of the water-in-oil or oil-in-water type and, depending on the size of the dispersed particles, they are called emulsions or microemulsions. Microemulsions, which are generally characterized by a dispersed particle size below 100 nm, can be obtained by reducing the surface tension with surfactants and have the advantage, compared with emulsions, of forming clear and stable compositions. In addition, these microemulsions are generally prepared from an anhydrous preconcentrate simply by mixing with water or with the gastric medium, practically without energy provision, to give a perfect dispersion of the active principle (Journal Dispersion Science and Technology 6 (3) pp. 317-337 (1985), or Progress in Surface and Membrane Science 12 pp. 405-477).

Thus microemulsions have been utilized in therapeutics for the purpose of promoting exchanges between the lipidic phases and the hydrophilic phases of biological media, thereby improving the passage of drugs through cell membranes.

A fenofibrate formulation in the form of a microemulsion preconcentrate was described in document WO 99/29300.

This formulation comprises a lipophilic phase in which the fenofibrate is solubilized, a hydrophilic phase and an emulsifying system.

With the exception of the formulation of Example 11, the formulations described in said document of the prior art all comprise an additional hydrophilic component, such as, in particular, ethanol and/or propylene glycol, which makes it possible to assure a good solubilization of the fenofibrate in the oily phase.

In general, the addition of ethanol and/or propylene glycol to an oily formulation facilitates the dissolution of the active principle in the lipophilic phase and thus makes it possible to avoid active principle crystallization phenomena. However, the use of such a hydrophilic compound is incompatible with the formulation of a drug in the form of sealed gelatin capsules or gelatin-based soft capsules, thereby limiting the value of such a formulation.

The present inventors have further found that fenofibrate-based formulations consisting solely of oil and an emulsifying system, such as the formulation described in Example 11 of the document cited above, generally exhibit a less than satisfactory stability from the point of view of their use as drugs, since it is possible to observe the appearance of fenofibrate crystals.

There is therefore a need, which has not been met hitherto, for a fenofibrate-based pharmaceutical formulation which is in the form of a preconcentrate capable of forming an oil-in-water microemulsion spontaneously on contact with an aqueous medium, has a very high bioavailability of the active principle and is stable over time, without requiring the use of an additional hydrophilic component which would limit its use for the preparation of sealed gelatin capsules or soft capsules.

It has been discovered that vitamin E acetate affords a durable stabilization over time of a fenofibrate-based preconcentrate whose lipophilic phase preferably consists of an oil based on esterified glycerol or propylene glycol, without the incorporation of any additional hydrophilic component and while at the same time preserving a very high bioavailability of the fenofibrate; it is this discovery which forms the basis of the present invention.

Thus, according to a first feature, the present invention relates to a pharmaceutical composition for the oral administration of fenofibrate in the form of a preconcentrate capable of forming an oil-in-water microemulsion spontaneously on contact with an aqueous medium, comprising:

a lipophilic phase preferably comprising an oil based on glycerol or propylene glycol, the latter preferably being totally esterified with medium-chain saturated fatty acids; and

an emulsifying system comprising:

a lipophilic surfactant preferably based on glycerol or propylene glycol partially esterified with medium-chain saturated fatty acids; and

a hydrophilic co-surfactant,

characterized in that it also comprises vitamin E acetate in a sufficient amount to stabilize said preconcentrate without the incorporation of an additional hydrophilic component.

This pharmaceutical composition is particularly advantageous insofar as, being devoid of a hydrophilic component, it is suitable for the preparation of a fenofibrate-based drug in the form of sealed gelatin capsules or soft capsules, with a prolonged stability and an excellent bioavailability. Within the framework of the present description, the expression “hydrophilic component” is understood as meaning any hydrophilic component other than water.

Furthermore, this pharmaceutical composition allows the spontaneous formation, without the provision of energy, of a microemulsion in the presence of an aqueous phase or in a gastric medium, the dispersed droplets generally having a size of between 10 and 50 nm.

This composition is particularly stable on storage at room temperature, no crystal precipitation or appearance phenomenon having been observed over a prolonged period of time.

In terms of pharmacokinetics, this pharmaceutical composition is particularly remarkable in that it makes it possible to limit the inter-individual variations and to reduce the effect associated with meals, while at the same time substantially increasing the bioavailability compared with the known formulations obtained by a dry process.

According to one particular characteristic of the invention, vitamin E acetate is present in the pharmaceutical composition in an amount such that the daily dose of vitamin E administered is at least about 100 International Units (IU), affording protection of the lipoproteins against oxidation.

Alternatively, part of the vitamin E administered can originate from the emulsifying system in the case where the latter comprises vitamin E TPGS (alpha-D-tocopherol polyethylene glycol succinate) as co-surfactant.

Particularly preferred pharmaceutical compositions within the framework of the present invention contain the following in relative amounts by weight:

a) 5 to 20% and preferably 6 to 9% of fenofibrate;

b₁) 0 to 69% and preferably 0 to 40% of oil;

b₂) 5 to 76% and preferably 5 to 50% of vitamin E acetate;

c₁) 3 to 69% and preferably 12 to 45% of surfactant;

c₂) 3 to 69% and preferably 12 to 45% of co-surfactant.

The oil forming part of the composition of the lipophilic phase is advantageously selected from triglycerides of medium-chain saturated fatty acids, especially triglycerides of C₈-C₁₂ saturated fatty acids and preferably those of C₈-C₁₀ saturated fatty acids. Medium-chain saturated fatty acids are understood as meaning C₈-C₁₂ saturated fatty acids, i.e. caprylic, capric and lauric acids. Among the commercially available compounds suitable for putting the invention into effect, there may be mentioned products sold under the name CAPTEX by ABITEC, especially the products called CAPTEX 300, CAPTEX 350 and CAPTEX 355, which are essentially triesters of glycerol with mixtures of caprylic, capric and lauric acids. Among the products of the same family which are normally obtained from copra oil, it is also possible to use MIGLYOL 812 marketed by HÜLS, which is a triglyceride of caprylic and capric acids. The oily phase can also comprise diesters of propylene glycol with C₈-C₁₂ fatty acids, for example a product marketed under the name CAPTEX 200 by ABITEC, which is an ester of caprylic and capric acids with propylene glycol.

The lipophilic phase can be devoid of oil if vitamin E acetate, which is itself lipophilic, is used in a substantial amount, for example of more than 25% by weight, based on the weight of the composition.

Among its essential constituents, the composition according to the invention also comprises an emulsifying system generally consisting of a lipophilic surfactant associated with a hydrophilic co-surfactant.

The lipophilic surfactant is preferably a non-ionic surfactant whose hydrophilic-lipophilic balance (HLB) is below 12. Among the compounds corresponding to these characteristics, preferred products are those resulting from the partial esterification of glycerol or propylene glycol with C₈-C₁₀ saturated fatty acids (caprylic and capric acids). Examples of products which can be used are those marketed under the names CAPMUL MCM, CAPMUL MCM C8 and CAPMUL MCM C10 by ABITEC, which are partial esters of glycerol with variable amounts of caprylic acid and capric acid. It is also possible to use the compound CAPTEX 200E6 (marketed by ABITEC), which is a caprylic or capric acid/propylene glycol ester reacted with ethylene oxide.

The co-surfactant present in the formulation is predominantly hydrophilic with an HLB above 12. Among the compounds capable of being used within the framework of the invention, there may be mentioned sorbitol/fatty acid esters copolymerized with ethylene oxide, which are generally called polysorbates.

Among the various products in this category, preference is given to polysorbate 80, marketed for example under the name TWEEN 80 by ICI or under the name MONTANOX 80 by SEPPIC, which is a sorbitol monooleate copolymerized with about 20 mol of ethylene oxide and having an HLB of about 15. Products which can also be used as co-surfactants are those resulting from the reaction of ethylene oxide with natural or hydrogenated castor oils, for example the products marketed by BASF under the name CREMOPHOR EL (obtained by reacting 35 mol of ethylene oxide with about 1 mol of castor oil and having an HLB of about 12 to 14) or CREMOPHOR RH40 (obtained by reacting 40 mol of ethylene oxide with about 1 mol of hydrogenated castor oil and having an HLB of about 14 to 16).

It should be pointed out that, within the framework of the present invention, the compound known as TPGS (alpha-D-tocopheryl polyethylene glycol 1000 succinate), which is of amphiphilic character with an HLB of between 15 and 19, can be used in the emulsifying system.

The use of this compound in the compositions according to the present invention is particularly advantageous insofar as this compound constitutes an additional source of vitamin E, which affords a good protection of the lipoproteins against oxidation as from a delivered daily dose of about 100 International Units (IU).

The formulation can optionally comprise additives commonly used in small amounts, for example flavorings or colors.

The main constituents (i.e. the active principle, the oil, the vitamin E acetate, the surfactant and the co-surfactant) must be present in relative proportions which make it possible to obtain a homogeneous phase capable of giving a microemulsion after dilution in an aqueous phase. Advantageously, the preferred proportions according to the invention make it possible to obtain, from the preconcentrate, a microemulsion which can include a high percentage of water in the presence of an aqueous phase, for example after dilution to 1/2500 or even after dilution to infinity. In addition, the proportions of the different components will advantageously be chosen to enable the daily delivery of a dose of at least about 100 IU of vitamin E, which is sufficient to ensure protection of the lipoproteins against oxidation.

As vitamin E acetate has a solvent effect on the active principle, it must be present in a sufficient amount for the solution to be stable and to avoid any risk of crystallization of the fenofibrate. If the amount of vitamin E acetate is large, i.e. represents e.g. more than 25% of the weight of the formulation, the oil can be omitted from the formulation, the fenofibrate then being solubilized by the vitamin E acetate.

The constituents with an emulsifying effect (surfactant and co-surfactant) must be present in a sufficient amount for the preconcentrate to form a stable microemulsion after dilution with an aqueous phase.

It has thus been determined on the basis of these principles that:

the active principle must be dissolved in the oily formulation and in an amount below the saturation limit, i.e., in practical terms, below about 20% by weight, preferably in the order of 6 to 9%, based on the weight of the formulation;

the amount of lipophilic phase (comprising the oil and the vitamin E acetate) and the amount of emulsifiers (i.e. the surfactant and the co-surfactant) must be in a weight ratio of between 1/15 and 5/1, preferably of between 1/5 and 2/1:

the oil and the vitamin E acetate constituting the lipophilic phase must be present in a weight ratio in the order of 0 to 15/1, preferably in the order of 0 to 10/1; and

the chosen amounts of surfactant and co-surfactant must be in a weight ratio of between 1/80 and 4/1, preferably of between 1/12 and 2/1.

In one of the preferred formulations of the invention, the ratio of the weight of oily phase to the weight of emulsifiers is about 1/2. the oily phase comprising similar amounts of oil and vitamin E acetate and the emulsifiers comprising approximately twice the amount of co-surfactant to surfactant, the active principle dissolved in the mixture representing about 6 to 9% of the weight of the final preparation.

In a second preferred formulation of the invention, the ratio of the weight of oily phase to the weight of emulsifying phase is 2/3, the oily phase comprising approximately twice the amount of vitamin E acetate to oil and the emulsifiers comprising similar amounts of surfactant and co-surfactant, the active principle representing about 7% of the preconcentrate.

Such formulations afford solutions which are particularly stable over time, under normal storage conditions, and capable of generating a microemulsion spontaneously when they are mixed with an aqueous phase, while at the same time comprising a dose of vitamin E which advantageously assures protection of the lipoproteins against oxidation.

According to a second feature, the present invention relates to a drug manufactured from the preconcentrated formulation described above.

In practical terms, the preconcentrate, in the form of a solution, will preferably be filled into sealed gelatin capsules or soft gelatin capsules which are soluble in the gastric medium to release the preconcentrate into the stomach and form the microemulsion in the presence of the gastric juice.

In another embodiment, the preconcentrate can be diluted immediately before use in a drink (water, fruit juice, etc.) to form a drinkable microemulsion. This form may be preferred by patients who have difficulty swallowing gelatin capsules or soft capsules and who wish to take drinkable medication.

The Examples which follow serve to illustrate the invention and must not be considered as implying a limitation, especially as regards the nature and amount of the various excipients.

Likewise, it is possible to modify the manufacturing procedure described for the Examples and to use a protocol which differs in the order of addition of the constituents or the agitation times, particularly if the equipment is of varying efficiency.

### EXAMPLE 1 TO 4

### Demonstration of the Effect of Vitamin E Acetate on the Solubilization of Fenofibrate in Oil

The solubility of fenofibrate in different formulations of oily phase or preconcentrate was evaluated in the presence or absence of vitamin E acetate and the following results were obtained:

| | | Solubility of fenofibrate in |
|---|---|---|
| | | mg/g of |
| EXAMPLE | | mixture |
| 1 | Captex 200 oil | 130 mg/g |
| | Captex 200/vit. E acetate 9/1 | 140 mg/g |
| | Captex 200/vit. E acetate 3/7 | 170 mg/g |
| 2 | Miglyol 812N oil | 100 mg/g |
| | Miglyol 812N/vit. E acetate 9/1 | 120 mg/g |
| | Miglyol 812N/vit. E acetate 3/7 | 145 mg/g |
| 3 | (Capmul MCM/Tween 80 1/1)/ | 108 mg/g |
| | Captex 200 50/50 | |
| | (Capmul MCM/Tween 80 1/1)/ | 123 mg/g |
| | (vit. E acetate/Captex 200 3/7) 50/50 | |
| 4 | (Capmul MCM/Tween 80 1/1)/ | 110 mg/g |
| | (vit. E acetate/Captex 200 3/7) 60/40 | |
| | (Capmul MCM/Tween 80 1/1)/ | 123 mg/g |
| | (vit. E acetate/Captex 200 3/7) 50/50 | |

(All the proportions are expressed as values by weight.)

Examples 1 and 2 clearly show that the incorporation of vitamin E acetate substantially increases the solubility of the fenofibrate in the oil.

Example 3 shows that the incorporation of vitamin E acetate also results in an increase in the solubility of fenofibrate in the presence of an emulsifying system.

Example 4 shows that an increase in the proportion of the pair (vit. E acetate/Captex 200) in the formulation comprising the emulsifiers improves the solubility of the fenofibrate. It may also be noted from a comparison of Examples 3 and 4 that the same solubility (108 and 110 mg/g) is obtained with less oily phase in the presence of vitamin E acetate.

### EXAMPLES 5 AND 6

The stability of preconcentrated formulations based on oil and an emulsifying system was evaluated in the presence or absence of vitamin E acetate and the following results were obtained:

| | Proportion | | Stability |
|---|---|---|---|
| | of | | at room |
| Example 5 | fenofibrate | Composition of oil | temperature |
| 56% of surfactants | 7% | 37% of Captex 355 | 7 months, then |
| 37% of oil | | | crystals |
| 56% of surfactants | 7% | 26% of Captex 355 + | still stable after |
| 37% of oil | | 11% of vit. E acetate | 18 months |
| | Proportion | | Stability |
| | of | | at room |
| Example 6 | fenofibrate | Composition of oil | temperature |
| 55% of surfactants | 8% | 37% of Captex 200 | 8 months, then |
| 37% of oil | | | crystals |
| 55% of surfactants | 8% | 26% of Captex 200 + | still stable after |
| 37% of oil | | 11% of vit. E acetate | 18 months |

These Examples clearly show that vitamin E acetate affords a substantial improvement in the stability of the preconcentrate at room temperature.

### EXAMPLE 7

### Preparation of a Drug According to the Invention in the Form of Soft Capsules

A mixture of 434 g of polysorbate 80 (TWEEN 80, HLB±15) and 217 g of a partial ester of glycerol with caprylic and capric acids (CAPMUL MCM, HLB±5 to 6) is prepared in a glass reactor with impeller-type agitation. The two products are mixed for 15 min and 139.5 g of vitamin E acetate are then added. When the mixture is of homogeneous appearance, 139.5 g of an ester of glycerol with caprylic and capric acids (CAPTEX 355) are added. Agitation is continued until a homogeneous phase is obtained, after which 70 g of finely powdered fenofibrate are added gradually at room temperature. The total mixing time is at least 3 to 5 hours to obtain a perfect solution. The resulting oily solution is filled into soft capsules each containing 1 g of solution, i.e. 70 mg of fenofibrate.

(In this formulation, the ratio of the amounts by weight of surfactant and co-surfactant is 1/2, that of the vitamin E acetate and the oil is 1/1 and that of the lipophilic phase and the surfactants is 3/7, the dose of vitamin E making it possible to assure a minimum daily dosage of 280 IU, administered in two doses.)

### EXAMPLES 8 to 22

### Examples of Formulations According to the Invention

A procedure analogous to Example 7 was followed in order to prepare Formulations 8 to 22, the qualitative and quantitative compositions of which are given in Table I (the amounts are expressed in percentages by weight and in the case of vitamin E are converted to the number of IU per g).

In this Table, the various constituents are as follows, being indicated without implying a limitation:

CAPMUL MCM is a partial ester of glycerol with caprylic and capric acids. This surfactant has an HLB of 5.5 to 6.

CAPMUL MCM C8 is a partial ester of glycerol with caprylic acid. This surfactant has an HLB in the order of 3 to 4.

TWEEN 80 is a polysorbate 80 or, more precisely, a sorbitol monooleate copolymerized with about 20 mol of ethylene oxide. This co-surfactant has an HLB of about 15.

Cremophor RH40 is the reaction product of hydrogenated castor oil with about 45 mol of ethylene oxide. This co-surfactant has an HLB of about 14 to 16.

Cremophor EL is the reaction product of castor oil with about 35 mol of ethylene oxide. This co-surfactant has an HLB of about 12 to 14.

Vitamin E TPGS (or TPGS) is a mixed ester of succinic acid with vitamin E and polyethylene glycol 1000.

Vit. E ac. is vitamin E acetate.

CAPTEX 200 is a diester of propylene glycol with caprylic and capric acids.

CAPTEX 355 is a triester of glycerol with caprylic acid (about 57%) and capric acid (about 40%).

The fenofibrate formulations according to the invention were tested to determine the bioavailability of the fenofibrate. The tests were conducted in parallel with dry fenofibrate formulations, which are known to exhibit the most favorable bioavailabilities at the present time. The tests were performed on non-fasted Sprague Dawley male rats, to which some of the compositions according to the invention were administered orally. By way of comparison, the test also included granules (fen. comp.) obtained according to the teaching of document FR 2 758 459, which, according to said document, correspond to the solid formulation giving the best bioavailability, and a formulation (LIP) of fenofibrate co-micronized with sodium laurylsulfate, as marketed in France under the mark LIPANTHYL 200M. The doses administered correspond to the administration of about 150 mg/kg of active principle.

The kinetics were established for each of the tests by taking 10 blood samples over a 72-hour period. The concentration of fenofibric acid in the sera was measured by HPLC (liquid chromatography) according to the known analytical methods. The values obtained made it possible to determine the values of the various parameters normally considered to represent the bioavailability of the active principle:

a) the standardized and extrapolated area under the curve (AUC), expressed in μg.h/ml;

b) the mean residence time (MRT), expressed in hours;

c) the maximum concentration (C max.) reached in the serum (expressed in μg/ml); and

d) the time after which the maximum concentration is obtained (T max.) (expressed in hours).

The results of the pharmacokinetic study are shown in Table II (fen. comp. is the first Comparative Example and LIP is the second Comparative Example).

TABLE I

| | % Surfactant (Sa) | | % Co-surfactant (co-Sa) | | | | | Vit. E | Oil | | | (Vit. E ac. + | |
|---|---|---|---|---|---|---|---|---|---|---|---|---|---|
| | c₁ | | c₂ | | | | | acetate | % | | | oil)/ | |
| | | Campul | | Cremo- | Cremo- | | Sa/ | % | b₁ | | Oil/vit. E | (Sa + co-Sa) | Feno- |
| | Capmul | MCM | Tween | phor | phor | | co-Sa | (IU)* | Captex | Captex | ac. | (b₂ + b₁)/ | fibrate |
| Ex. | MCM | C8 | 80 | RH 40 | EL | TPGS | c₁/c₂ | b₂ | 200 | 355 | b₁/b₂ | (c₁ + c₂) | % |
| 8 | 27.9 | | | 27.9 | | | 1 | 11.2 | | 26 | 7/3 | 2/3 | 7 |
| | | | | | | | | (112 IU) | | | | | |
| 9 | 27.9 | | 27.9 | | | | 1 | 11.16 | | 26.04 | 7/3 | 2/3 | 7 |
| | | | | | | | | (111 IU) | | | | | |
| 10 | | 27.9 | 27.9 | | | | 1 | 11.16 | | 26.04 | 7/3 | 2/3 | 7 |
| | | | | | | | | (111 IU) | | | | | |
| 11 | 23.25 | | 23.25 | | | | 1 | 13.95 | 32.55 | | 7/3 | 1 | 7 |
| | | | | | | | | (140 IU) | | | | | |
| 12 | 27.9 | | 27.9 | | | | 1 | 18.6 | 18.6 | | 1 | 2/3 | 7 |
| | | | | | | | | (186 IU) | | | | | |
| 13 | 18.6 | | 37.2 | | | | 1/2 | 11.16 | | 26.04 | 7/3 | 2/3 | 7 |
| | | | | | | | | (111 IU) | | | | | |
| 14 | 18.6 | | 37.2 | | | | 1/2 | 11.16 | 26.04 | | 7/3 | 2/3 | 7 |
| | | | | | | | | (111 IU) | | | | | |
| 15 | | 23.5 | | | 23.25 | | 1 | 13.95 | | 32.55 | 7/3 | 1 | 7 |
| | | | | | | | | (140 IU) | | | | | |
| 16 | 32.55 | | 21.7 | | | 10.85 | 1 | 27.9 | | | 0 | 3/7 | 7 |
| | | | | | | | | (321 IU)** | | | | | |
| 17 | 21.7 | | 28.93 | | | 14.47 | 1/2 | 27.9 | | | 0 | 3/7 | 7 |
| | | | | | | | | (335 IU)** | | | | | |
| 18 | 23.91 | | 31.89 | | | | 3/4 | 37.2 | | | 0 | 2/3 | 7 |
| | | | | | | | | (372 IU) | | | | | |
| 19 | 23.25 | | 15.5 | | | 7.75 | 1 | 46.5 | | | 0 | 1 | 7 |
| | | | | | | | | (495 IU)** | | | | | |
| 20 | 27.9 | | 18.6 | | | 9.3 | 1 | 37.2 | | | 0 | 2/3 | 7 |
| | | | | | | | | (408 IU)** | | | | | |
| 21 | 19.93 | | 26.57 | | | | 3/4 | 46.5 | | | 0 | 1 | 7 |
| | | | | | | | | (465 IU) | | | | | |
| 22 | 15.5 | | | 31 | | 13.95 | 0.35 | 5 | | 27.55 | 5.5 | 7/13 | 7 |
| | | | | | | | | (104 IU)** | | | | | |

*IU represents the number of units of vit. E introduced via the acetate and TPGS.
**with TPGS (contributes at a rate of 0.387 IU/mg)

TABLE II

| EX. | AUC | MRT | C max. | T max. |
|---|---|---|---|---|
| 7 | 8049 | 13.2 | 439 | 4 |
| 9 | 6902 | 13.4 | 361 | 12 |
| 10 | 6593 | 17.9 | 312 | 8 |
| 11 | 6298 | 11.8 | 393 | 4 |
| 12 | 6293 | 14.7 | 299 | 4 |
| 13 | 6169 | 13.8 | 325 | 8 |
| 14 | 6053 | 13.6 | 309 | 8 |
| 16 | 7166 | | | |
| 17 | 6872 | | | |
| 18 | 6695 | | | |
| 22 | 9820 | 13.8 | 454 | 4 |
| LIP | 3453 | 10.6 | 255 | 4 |
| Fen. comp. | 5303 | 15.5 | 288 | 4 |

Interpretation of these results, after correction of the dose administered, shows a very substantial improvement in the bioavailability of fenofibrate when it is formulated according to the invention: the area under the curve is appreciably increased, indicating that a larger amount of fenofibrate is actually absorbed.

In practical terms, the pharmaceutical composition can be packaged in doses each containing 50 to 80 mg of fenofibrate in the form of sealed gelatin capsules or soft capsules suitable for a dosage in the order of 1 to 3 doses per day, including an amount of vitamin E which assures a minimum daily dose of 100 IU.

To verify that the preconcentrate according to the invention is well dispersed in the gastric medium and that a microemulsion is obtained, the mean diameter of the droplets formed in the microemulsion after dilution of the preconcentrate was measured.

The sample was prepared for size measurement by diluting the pre-concentrate in water at 37° C. The mean diameter of the droplets is measured with a Coulter N4 granulometer after a stabilization time of 5 min, at an angle of 90°. All the formulations prepared give a clear stable microemulsion.

| Example | Droplet size (nm) | Dilution |
|---|---|---|
| Example 7 | 17.4 ± 12.4 | 1/250 |
| Example 9 | 37.9 ± 12.4 | 1/250 |
| Example 12 | 32.6 ± 7 | 1/250 |
| Example 14 | 33.1 ± 11 | 1/250 |
| Example 16 | 49 ± 16 | 1/2000 |
| Example 17 | 58 | 1/2500 |
| Example 18 | 37 ± 9.5 | 1/1000 |
| Example 19 | 53 ± 9 | 1/2500 |
| Example 20 | 42.8 ± 12 | 1/2000 |
| Example 21 | 52 ± 10 | 1/2500 |
| Example 22 | 30.3 ± 10 | 1/500 |

Diluting the preconcentrate in water or in an aqueous phase produces a clear solution consisting of a dispersion of droplets with a mean diameter of between 18 and 60 nm, these two factors being characteristic of a microemulsion. Furthermore, such a dispersion of the active principle favors absorption and affords a greater bioavailability than that previously obtained with the known formulations.

1. Pharmaceutical composition for the oral administration of fenofibrate in the form of a preconcentrate capable of forming an oil-in-water microemulsion spontaneously on contact with an aqueous medium, comprising:
a lipophilic phase preferably comprising an oil based on glycerol or propylene glycol, the latter preferably being totally esterified with medium-chain saturated fatty acids; and
an emulsifying system comprising:
a lipophilic surfactant preferably based on glycerol or propylene glycol partially esterified with medium-chain saturated fatty acids; and
a hydrophilic co-surfactant,
characterized in that it also comprises vitamin E acetate in a sufficient amount to stabilize said preconcentrate without the incorporation of an additional hydrophilic component.

2. Pharmaceutical composition according to claim 1, characterized in that it contains the following in relative amounts by weight:
a) 5 to 20% of fenofibrate;
b₁) 0 to 69% of oil;
b₂) 5 to 76% of vitamin E acetate;
c₁) 3 to 69% of surfactant;
c₂) 3 to 69% of co-surfactant.

3. Pharmaceutical composition according to claim 1 or 2, characterized in that it contains:
a) 6 to 9% of fenofibrate;
b₁) 0 to 40% of oil;
b₂) 5 to 50% of vitamin E acetate;
c₁) 12 to 45% of surfactant;
c₂) 12 to 45% of co-surfactant.

4. Pharmaceutical composition according to one of claims 1 to 3, characterized in that the emulsifying system comprises vitamin E TPGS (α-D-tocopherol polyethylene glycol 1000 succinate), preferably in an amount such that the daily dose of vitamin E administered is at least about 100 IU.

5. Pharmaceutical composition according to one of claims 1 to 4, characterized in that:
the components b₁ and b₂ are in a weight ratio b₁/b₂ of between 0 and 15/1, preferably of between 0 and 10/1;
the components c₁ and c₂ are in a weight ratio c₁/c₂ of between 1/80 and 4/1, preferably of between 1/12 and 2/1; and
the components b₁, b₂, c₁ and c₂ are such that the weight ratio (b₁+b₂)/(c₁+c₂) is between 1/15 and 5/1, preferably between 1/5 and 2/1.

6. Pharmaceutical composition according to one of claims 1 to 5, characterized in that the above-mentioned oil is a triglyceride of caprylic and capric acids.

7. Pharmaceutical composition according to one of claims 1 to 6, characterized in that the above-mentioned surfactant is a partial ester of glycerol and capric and caprylic acids.

8. Pharmaceutical composition according to one of claims 1 to 7, characterized in that the co-surfactant is a polysorbate, preferably polysorbate 80.

9. Pharmaceutical composition according to one of claims 1 to 7, characterized in that the co-surfactant is a reaction product of hydrogenated or non-hydrogenated castor oil with ethylene oxide such that the HLB is above 12.

10. Pharmaceutical composition according to one of claims 1 to 9, characterized in that it is presented in the form of sealed gelatin capsules or soft capsules.
